(19) United States
(12) Patent Application Publication
(10) Pub. No.: US 2011/0135697 A1
(43) Pub. Date: Jun. 9, 2011

(54) EDIBLE HOLOGRAPHIC SILK PRODUCTS

(75) Inventors: Fiorenzo Omenetto, Wakefield, MA (US); David L. Kaplan, Concord, MA (US)

(73) Assignee: Trustees of Tufts College, Medford, MA (US)

(21) Appl. No.: 12/999,087

(22) PCT Filed: Jun. 18, 2009
(86) PCT No.: PCT/US2009/047751

§ 371 Date: Jan. 28, 2011

Related U.S. Application Data
(60) Provisional application No. 61/073,609, filed on Jun. 18, 2008.
(60) Provisional application No. 61/088,063, filed on Aug. 12, 2008.

(51) Int. Cl. A61K 9/00; B32B 3/10; A23L 1/00; A23P 1/08

(52) U.S. Cl. 424/400; 428/195.1; 426/87; 426/383

(57) ABSTRACT

The present invention relates to edible silk holographic elements and methods for making the same. Edible silk holographic elements are used to label pharmaceuticals and foods, or may be formulated to deliver pharmaceuticals.

### RELATED APPLICATIONS

This application claims the benefit of U.S. Provisional Patent Applications Ser. No. 61/073,609, filed Jun. 18, 2008 and Ser. No. 61/088,063, filed Aug. 12, 2008, each incorporated fully herein.

This invention was made with government support under grants No. W911NF-07-1-0618 awarded by the Defense Advanced Research Projects Agency; No. FA9550-07-1-0079 awarded by the Air Force Office of Scientific Research; and No. EB002520 awarded by the National Institutes of Health. The U.S. government has certain rights in this invention.

### FIELD OF THE INVENTION

The present invention relates to silk tags, markers, or labels that provide holographic images. Specifically, nanopatterning allows the use of silk fibroin as a holographic medium, and the realization of surface relief holograms of high sophistication in a pure protein-based biopolymer that is entirely biocompatible, biodegradable, edible, and implantable.

### BACKGROUND

Source-of-product and counterfeit goods are of increasing concern for both safety and economic reasons. Regarding safety, a 2006 spinach E. coli outbreak killed 3 people and sickened more than 200. The spinach crisis was solved in about three weeks, in part because UPC codes on spinach bags enabled back-tracking the produce source. Most fruits and vegetables, however, do not provide such bar-codes or other means of identification. Because of a Salmonella outbreak in tomatoes in the spring of 2008, fresh produce will start bearing labels that identify the foods' country of origin. The Country of Origin Labeling Law (COOL) requires from Sep. 30, 2008, a verifiable audit trail from the retail store to the source and all food handlers along the way. The consumer, along with the grocer, has the right to know where the product comes from with field-to-fork information. Although there are labels available on some fruits and vegetables, the current paper-based stickers are often difficult to remove and should be removed before the product is used. Moreover, the current paper-based labels, although inexpensive to produce, are relatively easy to counterfeit.

Counterfeit goods also raise safety concerns. Injuries from overheating counterfeit cell phone batteries purchased right on Verizon store shelves sparked a 2004 recall by the Consumer Product Safety Commission (CPSC). Counterfeit trade is bringing a growing number of dangerous products into American homes: from smoke alarms with phony Underwriters Laboratories (UL) marks to bogus pharmaceutical pills stored under uncontrolled conditions and containing the wrong active ingredients. In 2006, over 14,000 shipments of counterfeit merchandise were confiscated. Regarding pharmaceuticals, the World Health Organization (WHO) estimates that 10% to 30% of medicines sold in developing countries may be counterfeit, and some studies conclude that the percentage may be even higher. Moreover, counterfeiting has increased as products are sold over the internet. For instance, samples of drug product obtained by the FDA from two of internet orders contained only talc and starch. According to the authentic drug manufacturer, these two samples displayed a valid lot number and were labeled with an expiration date of April 2007, but the correct expiration date for this lot number was actually March 2005. The FDA is working towards an Electronic pedigree (ePedigree) system to track drugs from factory to pharmacy. This technology may prevent the diversion or counterfeiting of drugs by allowing wholesalers and pharmacists to determine the identity and dosage of individual products. Some of the proposed anti-counterfeiting measures present concerns regarding privacy, or the possibility that drug manufactures may try to use anti-counterfeiting technologies to undermine legitimate parallel trade in medicines.

Further relating to safety, there are few mechanisms for identifying contamination or tampering with pharmaceuticals and foods. There is a need for an inexpensive but accurate indicator for freshness and safety. For example, there is a need for a label that could be placed directly on a food or package to warn a consumer that the food has contacted Salmonella, E. coli, or other dangerous contaminants; or on a pharmaceutical to indicate that the drug product has been stored in excessive heat or humidity or otherwise been tampered with.

Aside from safety concerns, counterfeiting has major economic ramifications. Counterfeit merchandise is estimated to cost legitimate businesses up to $250 billion in yearly sales. In 2003, the WHO cited estimates that the annual earnings of counterfeit drugs were over $32 billion. There are several technologies that may help combat this problem, such as radio frequency identification which uses electronic devices to track and identify items, such as pharmaceutical products, by assigning individual serial numbers to the containers holding each product. Such efforts illustrate the need for labels that are unique, and in the case of foods and pharmaceuticals, edible and biodegradable.

### SUMMARY OF THE INVENTION

An object of the present invention provides for an edible, biocompatible, biodegradable silk-embedded high resolution diffraction microrelief that confers a holographic image. An embodiment of the invention provides for a edible, biocompatible, biodegradable holographic label, a comprising silk fibroin protein, that may be placed directly on a product to provide identification. Another embodiment provides for an edible, biodegradable, biocompatible silk fibroin coating that surrounds a fruit or vegetable and also provides a holographic identification label, and may further preserve the product. In a related embodiment, the silk fibroin microrelief is organic.

Another embodiment provides for an edible, biocompatible, biodegradable, holographic label or mark comprising silk fibroin that may be applied to a pharmaceutical product, or may surround the entire pharmaceutical product, such as a pill or capsule, to provide identification and/or expiration dates. In a related embodiment, the silk hologram is incorporated into the wrapper or other packaging of an article of commerce, such as a shrink sleeve surrounding a bottle neck, or full-body sleeve.

Yet another embodiment of the invention provides for silk fibroin formulations that provide stability for small molecules, proteins, enzymes, organic and inorganic dyes, photoactive dyes, and the like, and also incorporate a holographic identification or information component. Such formulations may be used for administration of therapeutic formulations or implantation of diagnostic devices in which holograms provide identification and/or other information.

Another embodiment provides for programmed biosensors silk films that display a hologram or change color when they come into contact with bacteria or other contaminants.

The color change can either be associated to variation of the surface properties or variation of the bulk properties of the silk, or can be programmed as a function of the entrained biological components (i.e., small molecules, proteins, enzymes, organic and inorganic dyes, photoactive dyes and the like). Alternatively, the silk hologram is incorporated into currency.

Another embodiment, the silk hologram is part of an edible product, such as a vitamin or other nutritional supplement to provide identification as well as provide interest for the consumer, such as a day-of-the-week design for children's vitamins. Thus, in an embodiment the hologram provides information for the consumption of the film or graphic art to embellish and decorate the sheets of silk that can be consumed.

### BRIEF DESCRIPTION OF THE DRAWINGS

FIG. 1 shows a white light hologram realized in a 60 μm thick silk film. The film is 2.5 cm wide×1 cm high.

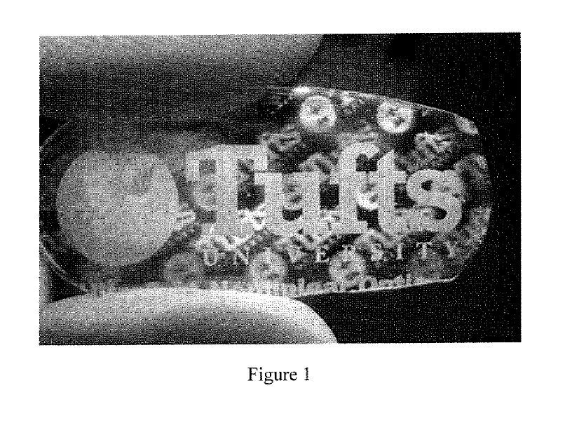

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

The present invention provides for silk as a holographic medium for the realization of surface relief holograms of high sophistication in a protein-based biopolymer that is entirely biocompatible, biodegradable, implantable, and edible.

Silk fibroin is a unique biopolymer that can be reconfigured from its native or synthesized states in various shapes and conformations. Silk fibroin protein has recently found uses well beyond textile and medical suture applications that have been the main modes of utilization in the past. For example, the generation of hydrogels (WO2005/012606; PCT/US08/65076; PCT/US08/65076), ultrathin films (WO2007/016524), thick films, conformal coatings (WO2005/000483; WO2005/123114), microspheres (PCT/US2007/020789), 3D porous matrices (WO2004/062697), combinations of the films, microspheres and porous matrices (PCT/US09/44117), solid blocks (WO2003/056297), microfluidic devices (PCT/US07/83646; PCT/US07/83634), electro-optical devices (PCT/US07/83639), and fibers with diameters ranging from the nanoscale (WO2004/0000915) to several centimeters (U.S. Pat. No. 6,902,932,) have been explored with implications in biomaterials and regenerative medicine (WO2006/042287; U.S. patent application Ser. No. 11/407,373; PCT/US08/55072). The holograph of the present invention may be used in conjunction with any of the above applications. The toughness of this natural fiber, unmatched in nature, confers impressive mechanical properties (both tensile and compressive) to silk-based materials which rival, if not exceed, most organic counterparts such as Kevlar or other polymeric materials.

Silk fibroin can be formed easily into mechanically robust films of thermodynamically-stable beta-sheets, with control of thicknesses from a few nanometers to hundreds of micrometers or more. These films may be formed by casting of purified silk fibroin solution which crystallizes upon exposure to air, humidity or dry nitrogen gas, as some examples, without the need for exogenous crosslinking reactions or post processing crosslinking for stabilization. The resulting hardened silk has mechanical properties, surface quality and transparency which are suited for use as optical substrates. See, e.g., PCT/US07/83600; PCT/US07/83620; PCT/US07/83605.

Silk fibroin has the ability to be patterned on the nanoscale. This property allows for silk to be used for the realization of sophisticated optical elements and other photonic components that range from waveguides, to optical fibers, 1D, 2D and 3D diffractive structures, reflectors, photonic crystals, nanocavities among others. See Lawrence et al., 9 (4) Biomacromol. 1214-20 (2008) (includes color photographs of silk holograms); Parker et al., 21 Adv. Mats. 1-5 (2009). Patterned nanostructures can be provided on the silk films or other structures manufactured. In one embodiment, the surface of the substrate may be smooth so as to provide a smooth silk biopolymer film, and a nanopattem may be machined on the surface of the silk film. The nanopattern may be machined using a laser, such as a femtosecond laser, nanoimprinting, or by other nanopattern machining techniques, including lithography techniques such as photolithography, electron beam lithography, soft lithography, and the like. Using such techniques, nanopattern features as small as 700 nm that are spaced less than 3 μm have been demonstrated. See PCT/US07/83620; PCT/US2008/082487. Indeed, nanopattemed features as small as 200 nm or less spaced less than 50 nm have been achieved. The very high resolution and conformal feature of surface patterning of silk allows for the fabrication of sophisticated diffraction structures and advanced holograms with more sophisticated security features and graphics, such as kinegrams.

Thus, nanopatterning allows the use of silk as a holographic medium and the realization of surface relief holograms and transmission holograms of high sophistication in a pure protein-based biopolymer that is entirely biocompatible, biodegradable, and implantable.

Surface relief holograms, which are now widely used, for instance, as security features on credit cards or on quality merchandise, can be replicated in silk allowing for unusual high definition images in an optically clear matrix. The possibility of achieving this in silk opens several opportunities by offering a new, low-cost, biocompatible substrate for holographic security and by bringing holographic security to the biomedical and pharmaceutical industries.

The ability to incorporate biological dopants in silk (such as pharmaceuticals, antibodies, enzyme, organic indicators, photoactive dyes among others) and maintain their biological viability and functionality under ordinary storage conditions allows for new modes of secure storage and branding of pharmaceuticals, or biological compounds by including the surface hologram on the silk matrix that incorporates the biological or pharmaceutical substance of interest. See, e.g., PCT/US09/44117; Lawrence et al., 2008. This is achievable because silk fibroin may be processed in a water-based system under ambient temperature and pressure conditions.

Moreover, silk holograms provide for color and interest without the use of chemical dyes. Indeed, silk fibroin films provide the capability of producing a greater variety of colors beyond the few that have regulatory approval—especially “rainbow-like” effects produced by the juxtaposition of multiple colors of gradually varying wavelength.

The ability to realize holograms in silk allow for a number of applications, including pharmaceutical branding, food labeling, therapeutic printed silk, and novelty items as edible products, including dosage forms in any of a wide variety of shapes and configurations, that have a stable microrelief with stability that can be controlled, and that conveys information such as visual holographic images and effects.

Regarding pharmaceutical branding, silk films can also be made to include pharmaceutical components turning the films into ingestible drugs. This is possible based on previous results that have shown that silk is a completely organic, ingestible, non toxic biopolymer in combination with the fact that it is possible to entrain biological compounds in the films while maintaining their viability. See, e.g., PCT/US07/83620. Further, the silk will degrade due to proteolytic activity in the body. See, e.g., PCT/US09/44117. Release and degradation rates may be controlled by manipulating the beta-sheet structure and layering and/or with the addition of excipients or bioerodable, biocompatible polymers.

Once the drug is incorporated in the silk film, the latter can be surface-patterned easily to contain a hologram that will be available for branding, for example to guarantee the authenticity of the drug point of origin and manufacturing. Individualized information on the pharmaceutical can be impressed on any single dose along with the hologram, including the expiration date or the name of patient. The dose may also include selective codes or covert identifiers for tracking or security purposes that may lack clear designation, requiring magnification, a change in environmental conditions, or particular light sources for viewing. Aside from tracking and security, such covert markings may be employed in double blind studies or clinical trials. The demonstrated capacity of the silk to be patterned with resolution down to less than 30 nm and to be able to faithfully replicate features on the micro and nanoscales enables sophisticated security to be incorporated in the pharmaceutical compound with applications that go beyond white light holograms but incorporate technically advanced security devices such as Kinegrams, Pixelgrams, Exelgrams, Fourier Transform structures, or photonic bandgap lattices.

To warrant survivability of labile compounds, the holographic pharmaceuticals may be impressed on the surface of the film via the casting of the silk solution on a master surfaces—depending on the pharmaceutical compound embossing might be suitable provided that the pharmaceutical can survive exposure to a few seconds of moderate heat exposure. Thus, the embossing could be done in situ (on the pill, hard capsule, soft capsule, drug, and the like) depending on the stability of the material, or on thin films first that are then wrapped, coated or stuck onto the pill or capsule post-embossing.

For coatings, silk fibroin can be doped with biocompatible plasticizers, such as glycerol, that maintain the optical features while conferring significant flexibility and elasticity to the film or coating. This feature provides a simple means to pre-emboss and then wrap or coat onto pills after the embossing process, or provide labels for food products. The glycerol is fully biocompatible and edible as well. Levels can vary form 0% to 50% of the silk formulation, depending on the degree of flexibility desired. Levels above 50% can also be used, although the films will be much less mechanically robust. See U.S. patent application Ser. No. 61/104,135.

Indeed, the choice of plasticizer and the relative portions may be adjusted to control the response of the microrelief over time to humidity. Oils and waxes with varying melting points admixed to this layer provide control over the response of the microrelief over time to temperature. Fading or change of color (due to a change in the reconstruction angle) of the visual image or effect produced by the microrelief provides a visual indication of the environmental history of the dosage form and its integrity. In addition to glycerol, suitable waxes include paraffin (a low melting point) and carnuba (a high melting point); suitable hygroscopic plasticizers include sugars such as dextrose (highly hygroscopic) and propyleneglycol. Hence, in addition to identification information, the structural integrity of the label may be “programmed” to change over time such that the label changes in coordination with, for example, either the drugs expiration date or the patient's treatment period.

Regarding therapeutic printed silk, in the same way that silk film sheets can be made to contain pharmaceutical compounds, other therapeutic compounds such as vitamins or dietary supplements can be included in the silk, as mentioned above. In this way, printed individual multi-day regimens for adults and children alike may improve compliance. Possible products are sheets or books with tear-away portions or pages that include the daily dosage of therapeutic, puzzles where parts are consumed according to a game, edible cards and letters, and many related toys and consumer items. The addition of surface structuring, coloring and suitable flavorings as are known in the art, adds possibilities for branding, embellishment and easy recognition, including olfactory enticements.

Food labeling provides a particularly suitable application of the present invention. For example, not only could a spinach bag carry the silk hologram label, the spinach itself might be labeled with the edible microrelief. Because the label is small and edible, it need not be removed before cooking or consumption. Fruits such as apples and tomatoes may bear a label, or may be surrounded by a microrelief-bearing silk film. In that regard, fruit can be dipped or otherwise introduced into silk fibroin solution, then dried by air or gas. Such process might provide both stability to the food product as well authentication regarding origin and whether the food is certified organic.

Silk labels, unlike current paper-based labels, may themselves be certified organic. Silk fibroin produced by silkworms, such as Bombyx mori, is the most common and represents an earth-friendly, renewable resource. Silkworm cocoons are commercially available from silkworms fed on U.S. Dept. of Agriculture Certified Organic mulberry leaves. Additionally, vegetarian or “peace silk”, from cocoons from which silk moths emerge, yield silk fibroin suitable for use in the silk holograms of the instant invention. The organic silk fibroin may be prepared from organic-fed silkworm cocoons using water- and salts-based techniques disclosed, for example, in U.S. patent application Ser. No. 11/247,358, WO/2005/012606, and PCT/US07/83605. Hence, the edible hologram label that identifies a food as certified organic may itself, when organic silk standards are finalized, be certified organic.

Moreover, the silk labels may have biosensor capabilities such that they are ‘edible optics’ that can be used as sensors for E. coli, Salmonella, and other potentially deadly contaminants. For example, the sensors thus display a hologram warning or change color when they come into contact with unwanted bacteria. Methods for constructing silk biosensor have been discussed, see, e.g., PCT/US07/83620; Lawrence et al., 2008; Parker et al., 2009. Inexpensive silk-based sensors that resemble transparent pieces of thin plastic may be tossed into a bag of produce, or even used to make the produce bags themselves. Films made from optic silks could also be used to coat salad tongs in a restaurant, or even be shredded and sprinkled on top of food.

Novelty products allow for a number of images both 2-D and 3-D and combinations thereof to be manufactured in silk. The non-toxic nature of silk provides an ideal material substrate for the incorporation of high quality holographic images without introducing any toxic component or any chemical processing. The holographic silk films can be used as stand alone components or can be used as biocompatible nontoxic coatings that can provide the brilliant graphic designs obtainable with holograms.

Under the same principles, edible toys, games and cards can be made with silk taking advantage of the properties of the material. Further, these same films can be doped with colorings (e.g., food color or other biocompatible dyes), flavors, vitamins, nutrients of various sources and related materials. Thus, aside from embossing for tracking films based on encoded information, the pills can also be encoded based on ‘olfactory’ signatures. This allows rapid screening via gas chromatography-mass spectroscopy to identify fingerprints against a library or data base for the information on the pharmaceutical.

Additional applications employ the same concepts outlined above, and are applicable in similar ways for the tracking of textiles, clothes, chemicals, fertilizers, and almost any consumer product where human contact with a biocompatible coating would be useful, optionally edible, and environmental friendly in both production and disposal. This may also apply to building supplies, paints, plumbing and electrical parts, art work, museum items, and related works of art.

### EXAMPLES

### Example 1

### Silk Hologram by Casting Silk Fibroin Solution on Appropriate Surface

Production of the silk fibroin solution begins with the purification of harvested B. mori cocoons. Sericin, a water-soluble glycoprotein which binds fibroin filaments, is removed from the fibroin strands by boiling the cocoons in a 0.02 M aqueous solution of Na₂CO₃ for 45 min. Upon completion of this step, the remaining fibroin bundle is rinsed thoroughly in Milli-Q water and allowed to dry overnight.

The dry fibroin bundle is then dissolved in a 9.3 M aqueous solution of LiBr at 60° C. for 4 hr. The LiBr salt is then extracted from the solution over the course of three days, through a water-based dialysis process. The resulting solution is extracted from the dialysis cassette (e.g., Slide-a-Lyzer, Pierce, MWCO 3.5K) and remaining particulates are removed through centrifugation and syringe based micro-filtration (5 μm pore size, Millipore Inc., Bedford, Mass.). This process enables the production of 8%-10% w/v silk fibroin solution of excellent quality and stability. The purification step is important for the generation of high quality optical films with maximized transparency and, consequently, minimized scattering. Films can also be generated from silk solutions at higher or lower percent protein.

The patterning of silk fibroin films can be achieved, for example, by a modified soft-lithography casting process or through a hot embossing process. See also, Lawrence et al., 2008.

For example, during the casting process, 200 μL to 1 mL of silk fibroin solution is deposited onto a clean, dry master. This solution is then allowed to crystallize in free air at ambient temperature and pressure. Under these settings, dry films are produced after approximately 16 hours. Alternative post-processing techniques (such as water vapor annealing or exposure to methanol) can be used to shorten the time necessary for beta-sheet film formation.

Removal of the film can be accomplished by loosening at one corner of the master and subsequent levering off using a thin razor blade or scalpel. Surfactants can also be used to help in the removal process from the master.

Once the film has been removed from the master, the silk fibroin can be further cross-linked through exposure to vacuum-induced methanol vapor (100% methanol at 26 mmHg), or water vapor (less than 10 mmHg-3 mmHg), for a period of 24 hours to 36 hours. This step is optional, based on the use for the films. Other post processing techniques can be used to confer the desired structural stability to the film.

In the hot embossing procedure, the mask is slowly heated to temperatures above 120° C. This temperature is generally optimized as a function of the particular film that is being used. The temperature is generally a function of parameters such as film thickness, film post-processing and imprint size.

1. A pharmaceutical bearing a silk-embedded high resolution diffraction relief which confers a holographic image on said pharmaceutical.

2. A food product bearing a silk-embedded high resolution diffraction relief which confers a holographic image on said food product.

3. A package that bears a silk-embedded high resolution diffraction relief which confers a holographic image on said package.

4. An edible novelty bearing a silk-embedded high resolution diffraction relief which confers a holographic image on said edible novelty.

5. A nutraceutical bearing a silk-embedded high resolution diffraction relief which confers a holographic image on said nutraceutical.

6. A method of preparing an edible product having a high resolution diffraction relief which confers a holographic image on said product comprising the steps of contacting a silk fibroin polymer with a high resolution diffraction relief mold, allowing the silk fibroin to harden, and removing the silk fibroin from the mold.

7. The food product of claim 2, wherein the silk-embedded high resolution diffraction relief which confers a holographic image on said food product comprises a biosensor that indicates whether the food is contaminated.
